(19) United States
(12) Patent Application Publication
(10) Pub. No.: US 2016/0106328 A1
(43) Pub. Date: Apr. 21, 2016

(54) DETERMINING ARTERIAL PULSE TRANSIT TIME FROM TIME-SERIES SIGNALS OBTAINED AT PROXIMAL AND DISTAL ARTERIAL SITES

(71) Applicant: Xerox Corporation, Norwalk, CT (US)

(72) Inventors: Lalit Keshav MESTHA, Fairport, NY (US); Survi KYAL, Rochester, NY (US)

(21) Appl. No.: 14/515,618

(22) Filed: Oct. 16, 2014

(51) Int. Cl. A61B 5/021; A61B 5/0295; A61B 5/02; A61B 5/026; A61B 5/00

(52) U.S. Cl. CPC A61B 5/02125; A61B 5/0261; A61B 5/7278; A61B 5/02028; A61B 5/0295; A61B 5/4884; A61B 5/7253; A61B 5/746; A61B 5/747; A61B 5/743; A61B 5/681; A61B 5/6898; A61B 5/0022; A61B 5/7225; A61B 5/02007; A61B 2560/0475; A61B 2562/0238

(57) ABSTRACT

What is disclosed is a system and method for determining arterial pulse transit time (PTT) for a subject. In one embodiment, time-series signals are received for each of a proximal and distal arterial site of a subject's body which represent blood volume changes in the microvascular tissue at each site. A proximal and distal analytic signal is obtained which has a first component being a waveform of the respective time-series signal and a second component being a transform of the respective waveform. A phase function is determined for the first and second components of each analytic signal. The phase function obtained for the proximal waveform is then subtracted from the phase function obtained for the distal waveform to get a phase difference. The phase difference is analyzed with the subject's heart rate to determine an arterial pulse wave transit time between the two proximal and distal sites.

### TECHNICAL FIELD

The present invention is directed to systems and methods for determining the time it takes for an arterial pulse pressure wave to travel from one arterial site on a patient's body to another (downstream) arterial site such that various arterial and cardiac functions can be assessed in real-time and on a continuous basis.

### BACKGROUND

Pulse Transit Time (PTT) can be the time it takes an arterial pulse pressure wave to travel from a proximal arterial site to a distal (downstream) arterial site. PTT is a function of wave velocity which, in turn, is a function of pressure, vessel diameter, and blood density. PTT can be useful by medical professional diagnosing cardiac stress, heart disease, and peripheral vascular disease in diabetic patients. There are many challenges in estimating PTT from signals obtained from a proximal and distal arterial site. Sophisticated systems and methods for obtaining PTT are increasingly needed in the medical arts. The present invention is directed toward this effort.

Accordingly, what is needed in this art is a system and method for determining the time it takes for an arterial pulse pressure wave to travel from a proximal arterial site on a patient's body to a distal arterial site such that various arterial and cardiac functions can be assessed in real-time and on a continuous basis.

### INCORPORATED REFERENCES

The following U.S. Patents, U.S. Patent Applications, and Publications are incorporated herein in their entirety by reference.

“Deriving Arterial Pulse Transit Time From A Source Video Image”, U.S. patent application Ser. No. 13/401,286, by Mestha et al.

“System And Method For Determining Video-Based Pulse Transit Time With Time-Series Signals”, U.S. patent application Ser. No. 14/026,739, by Mestha et al.

“System And Method For Determining Arterial Pulse Wave Transit Time”, U.S. patent application Ser. No. 14/204,397, by Mestha et al.

“Continuous Cardiac Pulse Rate Estimation From Multi-Channel Source Video Data With Mid-Point Stitching”, U.S. patent application Ser. No. 13/871,728, by Kyal et al.

“Continuous Cardiac Signal Generation From A Video Of A Subject Being Monitored For Cardiac Function”, U.S. patent application Ser. No. 13/871,766, by Kyal et al.

### BRIEF SUMMARY

What is disclosed is a system and method for determining the time it takes for an arterial pulse pressure wave to travel from a proximal arterial site on a patient's body to a distal arterial site such that various arterial and cardiac functions can be assessed in real-time and on a continuous basis. In one embodiment, the present method for determining arterial pulse wave transit time (PTT) for a subject involves the following. Time-series signals are received for each of a proximal and distal arterial site of a subject's body. The received time-series signals represent blood volume changes in the subject's microvascular subcutaneous tissue at each of the arterial sites. Then, for the proximal and distal time-series signals, a proximal and distal analytic signal is obtained. Each of the analytic signals comprises a first and second component. The first component is a waveform of the respective (proximal or distal) time-series signal and the second component is a transform (such as the Hilbert transform) of that waveform. A phase function is then determined with respect to time for the first and second components of each of the proximal and distal analytic signals. The phase function determined for the proximal waveform is subtracted from the phase function determined for the distal waveform to obtain a phase difference. The phase difference is then processed, in a manner more fully disclosed herein, with the subject's heart rate to determine an arterial pulse wave transit time between the proximal and distal arterial sites. The arterial pulse wave transit time is then communicated to a display device.

Features and advantages of the above-described method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows a subject of interest's right arm extremity clutching a pole to illustrate a proximal and distal arterial site in a subject's arm;

FIG. 2 is a flow diagram which illustrates one example embodiment of the present method for determining arterial pulse wave transit time for a subject in accordance with the teachings disclosed herein;

FIG. 3 is a block diagram of an example networked signal processing system wherein various aspects of the present method as described with respect to the flow diagram of FIG. 2 are implemented;

FIG. 4 shows simulated proximal and distal waveforms;

FIG. 5 shows the filtered proximal signal and the Hilbert transform of the filtered proximal signal; and

FIG. 6 shows the filtered distal (delayed) signal and the Hilbert transform of the filtered distal signal.

### DETAILED DESCRIPTION

What is disclosed is a system and method for determining arterial pulse wave transit time for a subject in real-time and on a continuous basis.

### Non-Limiting Definitions

A “subject” refers to a living being. Although the term “person” or “patient” may be used throughout this disclosure, it should be appreciated that the subject may be something other than a human such as, for example, a primate. Therefore, the use of such terms is not to be viewed as limiting the scope of the appended claims strictly to human subjects.

“Proximal” is from the Latin proximus: meaning nearest to. A proximal point in the arterial network is a point which is closer to the heart.

“Distal” is from the Latin distare: meaning away from. For the purposes hereof, a distal point in the arterial network is a point which is downstream from the proximal point in the arterial network.

An “arterial pulse pressure wave” is a wave created throughout the vascular system when the left ventricle of the heart contracts and pushes a volume of blood into the aorta. This generates a perturbation that travels into the arterial network. An arterial pulse pressure wave has two primary components, i.e., a forward traveling wave and a reflected wave returning back from the peripheral vascular network. The actual pressure in the aorta is the sum of the forward wave and the reflected wave.

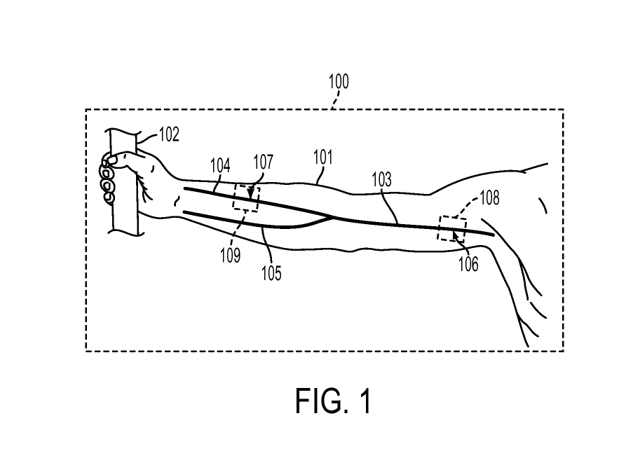

The “arterial pulse wave transit time” or simply pulse transit time (PTT) is the time it takes for an arterial pulse pressure wave to travel from a proximal arterial site to distal arterial site on the subject's body. PTT can be used for a variety of medical determinations including blood pressure, blood vessel dilation over time, location of a blood flow blockage, and blood flow velocity. FIG. 1 shows a subject's right arm 101 extended outward and clutching a section of a vertical pole 102. Brachial artery 103 extends down the arm and branches into the radial and ulnar arteries at 104 and 105, respectively. Point 106 in the brachial artery is proximal to point 107 in the radial artery. In FIG. 1, the PTT is the time it takes the arterial pulse pressure wave to travel from point 106 in proximal arterial site 108 to point 107 in distal arterial site 109. In accordance with the methods disclosed herein, a time-series signal is received for each of the proximal and distal arterial sites.

A “time-series signal” contains frequency components that represent blood volume changes in the microvascular subcutaneous tissue at a given proximal or distal arterial site. Time-series signals can be derived from a contact-based photoplethysmographic (PPG) device or from processing image frames acquired by a video imaging device that is capable of registering a videoplethysmographic (PPG) signal on a least one imaging channel use to capture that video. Methods for obtaining a time-series signal from video image frames and for extracting VPG signals from the time-series signals are disclosed in the incorporated references. The video imaging device can be a contact-based video camera, a non-contact-based video camera, a RGB camera, a multi-spectral camera, a hyperspectral camera, and a hybrid camera comprising any combination hereof.

“Receiving time-series signals” is intended to be widely construed and means retrieving, capturing, acquiring, or otherwise obtaining time-series signals corresponding to a proximal and distal arterial site of the body for processing in accordance with the methods disclosed herein. The time-series signals can be retrieved from a memory or storage of the device used to acquire those signals. Time-series signals can be retrieved from a media such as a CDROM or DVD, or can be received from a remote device over a network. Time-series signals may be downloaded from a web-based system or application which makes such signals available for processing.

“Obtaining an analytic signal” can be effectuated as follows. A proximal analytic signal is obtained from the received time-series signal as follows:

zₚ(t)=xₚ(t)+jyₚ(t) (1)

where zₚ(t) is the proximal (subscript ‘p’) analytic signal comprising a first component which is a proximal waveform xₚ(t) of the received time-series signal corresponding to the proximal arterial site and a second component yₚ(t) which is the Hilbert transform of the proximal waveform, and j=\sqrt{−1} is a complex number. It should be noted that, the second component of the analytic signal is a version of the original waveform but in phase quadrature to the original waveform (i.e., contains a 90° phase shift for every component). Hence, the component xₚ(t) is the in-phase component and yₚ(t) is the quadrature component.

The distal analytic signal can be similarly written as:

z_{d}(t)=x_{d}(t)+jy_{d}(t) (2)

It should be appreciated that a Hilbert transformed waveform has the same amplitude and frequency content as the original (proximal or distal) waveform and includes phase information that depends on the phase of the original data. The reader is directed to Chapter 15 of the textbook: “Handbook of Formulas and Tables for Signal Processing”, CRC Press, 1^{st} Ed. (1998), ISBN-13: 978-0849385797, which is incorporated herein in its entirety by reference.

“Determining a phase function with respect to time” can be effectuated as follows. Given the relationship of Eq. (1), the phase function with respect to time (i.e., the instantaneous phase angle) can be written as:

$${φ}_{p} ( t ) = arctan [ \frac{{y}_{p} ( t )}{{x}_{p} ( t )} ] \qquad ( 3 )$$

Given Eq. (2), the phase function with respect to time can be similarly written as:

$${φ}_{d} ( t ) = arctan [ \frac{{y}_{d} ( t )}{{x}_{d} ( t )} ] \qquad ( 4 )$$

The “phase difference” is obtained as follows:

dφ=φₚ(t)−φ_{d}(t) (5)

In certain applications, phase unwrapping may be required for φₚ(t) and φ_{d}(t).

“Processing the phase difference” to obtain the arterial pulse wave transit time can be determined by:

$$PTT = \frac{d φ}{{f}_{HR}} \qquad ( 6 )$$

where f_{HR} is the subject's heart rate (cardiac frequency in radians per second).

It should be appreciated that the steps of “determining”, “analyzing”, “obtaining”, “subtracting” and “processing”, as used herein, include the application of various signal processing and mathematical operations applied to data and signals, according to any specific context or for any specific purpose. It should be appreciated that such steps may be facilitated or otherwise effectuated by a microprocessor executing machine readable program instructions retrieved from a memory or storage device.

### Flow Diagram of One Embodiment

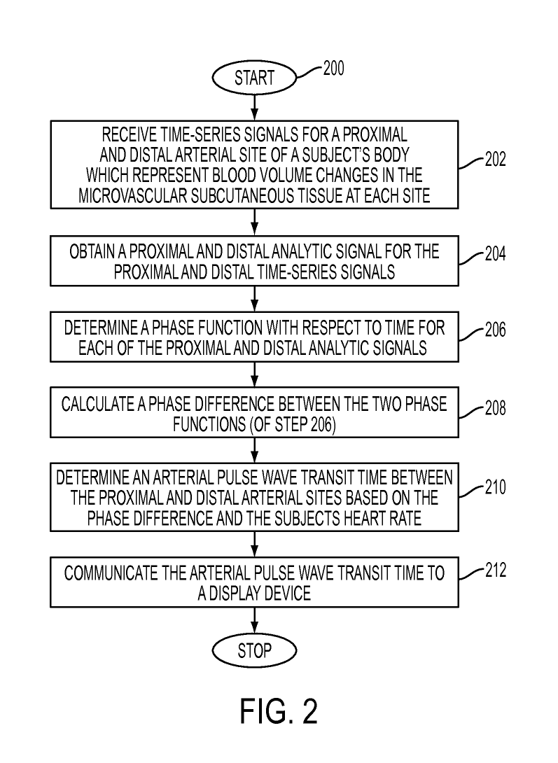

Reference is now being made to the flow diagram of FIG. 2 which illustrates one example embodiment of the present method for determining arterial pulse wave transit time for a subject. Flow processing begins at step 200 and immediately proceeds to step 202.

At step 202, receive time-series signals for a proximal and distal arterial site of a subject's body which represent blood volume changes in the microvascular subcutaneous tissue at each site. Example proximal and distal arterial sites are shown at 108 and 109, respectively, in FIG. 1.

At step 204, obtain a proximal and distal analytic signal for the proximal and distal time-series signals. Each of the proximal and distal analytic signals comprises a first component being a waveform of the respective time-series signal and a second component being a Hilbert transform of that waveform. Analytic signals for each of the proximal and distal arterial sites are given in Eqs. (1) and (2).

At step 206, determine a phase function with respect to time for each of the proximal and distal analytic signals. One method for determining the phase functions for each of the proximal and distal waveforms is given in Eqs. (3) and (4).

At step 208, calculate a phase difference between the two phase functions (of step 206). This is given in Eq. (5).

At step 210, determine an arterial pulse wave transit time between the proximal and distal arterial sites based on the phase difference and the subject's heart rate. One embodiment for PTT determination is given in Eq. (6).

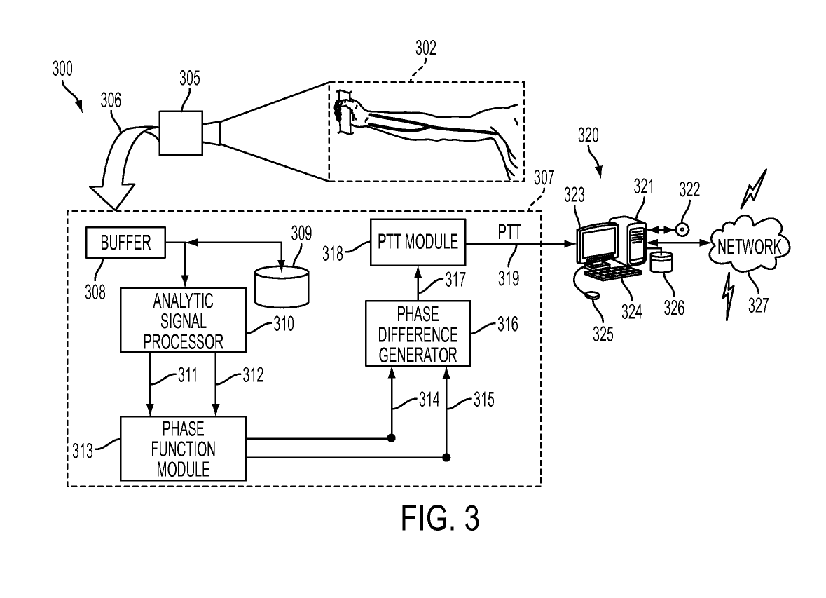

At step 212, communicate the arterial pulse wave transit time to a display device. One example display device is shown at 323 in FIG. 3. In other embodiments, the subject's arterial pulse wave transit time is communicated to a memory, a storage device, a handheld wireless device, a handheld cellular device, and a remote device over a network or an electronic medical record (EMR). An alert signal may be initiated and a signal may be sent to a medical professional.

It should be appreciated that the flow diagrams depicted herein are illustrative. One or more of the operations illustrated in the flow diagrams may be performed in a differing order. Other operations may be added, modified, enhanced, or consolidated. Variations thereof are intended to fall within the scope of the appended claims.

### Block Diagram of Signal Processing System

Reference is now being made to FIG. 3 which illustrates a block diagram of one example signal processing system 300 for determining arterial pulse wave transit time for a subject in accordance with the embodiment described with respect to the flow diagrams of FIG. 2.

In the embodiment of FIG. 3, video imaging device 305 is shown acquiring video of a subject's arm in image frame 302. Proximal and distal time-series signals (collectively at 306) obtained from processing video image frames acquired by the video imaging device are communicated to a Signal Processing System 307 shown comprising, in part, a Buffer 308 for buffering the received time-series signals for processing. The Buffer may further utilize storage device 309 to save/retrieve various formulas, mathematical representations, and the like, as needed to process time-series signals in a manner disclosed herein.

Analytic Signal Processor 310 processes the time-series signals to obtain proximal and distal analytic signal, at 311 and 312 respectively, each comprising a first component and second component. Proximal analytic signal 311 has a first component that is a waveform of the proximal time-series signal 306 and a second component that is a transform of that waveform. Likewise, distal analytic signal 312 has a first component that is a waveform of the distal time-series signal and a second component that is a transform of that waveform. Phase Function Module 313 receives the proximal and distal analytic signals and computes a phase function with respect to time, φₚ(t) and φ_{d}(t), at 314 and 315 respectively. Phase Difference Generator 316 receives the proximal and distal phase functions and computes a phase difference dφ, at 317. PTT Module 318 computes the arterial pulse wave transit time 319 for the subject. The generated arterial pulse transit time is communicated to networked computer system 320.

Workstation 320 reads/writes to computer readable media 322 such as a floppy disk, optical disk, CD-ROM, DVD, magnetic tape, etc. Case 321 houses a motherboard with a processor and memory, a network card, graphics card, and the like, and other software and hardware. The workstation includes a user interface which, in this embodiment, comprises display 323 such as a CRT, LCD, touch screen, etc., a keyboard 324 and a mouse 325. A user may use the keyboard and/or mouse to identify signal components of interest, and perform other functionality such as, for example, average multiple received proximal or distal time-series signals to obtain a composite proximal or distal time-series signal; discard any of received time-series signals as not being of interest; and/or perform a weighted averaging on any of the received time-series signals based on a statistical analysis. A user may further use the user interface of the workstation 320 to detrend any of the received time-series signals to remove non-stationary components; filter any of the received time-series signals to restrict frequencies of interest; perform peak detection on any of the received time-series signals; and normalize any of the received time-series signals to have a zero-mean unit variance. The user interface of the workstation may further be used to set parameters, view results, and the like. It should be appreciated that the workstation has an operating system and other specialized software configured to display a variety of numeric values, text, scroll bars, pull-down menus with user selectable options, and the like, for entering, selecting, or modifying information displayed on display device 323. Various portions of the received time-series signals may be communicated to workstation 320 for processing and stored to storage device 326 through pathways not shown. Workstation 320 is shown having been placed in communication with one or more remote devices of network 327 via a communications interface internal to case 321. Although shown as a desktop computer, it should be appreciated that computer workstation 320 can be any of a laptop, mainframe, server, or a special purpose computer such as an ASIC, circuit board, dedicated processor, or the like.

Some or all of the functionality performed by any of the modules and processing units of system 307 can be performed, in whole or in part, by workstation 320. Any of the modules and processing units of FIG. 3 can be placed in communication with storage devices 309, 322 and 326 and may store/retrieve therefrom data, variables, records, parameters, functions, machine readable/executable program instructions required to perform their intended functions. Each of the modules of system 307 may be placed in communication with one or more devices over network 327.

It should be appreciated that various modules may designate one or more components which may, in turn, comprise software and/or hardware designed to perform their intended functions. A plurality of modules may collectively perform a single function. Each module may have a specialized processor capable of executing machine readable program instructions. A module may comprise a single piece of hardware such as an ASIC, electronic circuit, or special purpose processor. A plurality of modules may be executed by either a single special purpose computer system or a plurality of special purpose computer systems operating in parallel. Connections between modules include both physical and logical connections. Modules may further include one or more software/hardware modules which may further comprise an operating system, drivers, device controllers, and other apparatuses some or all of which may be connected via a network.

### Performance Results

The methods disclosed herein was implemented for (a) synthetic signals and (b) for real waveforms obtained from human subjects with a video camera.

PTT estimation was simulated for a composite signal synthesized with three harmonics, low frequency modulation and time varying phase variation. A fundamental frequency of f=1 Hz was chosen in an effort to emulate the heart rate frequency of 60 beats per minute (bpm).

The proximal signal is:

xₚ(t)=−((sin(2πft)−0.2 sin(2π2ft)−0.2 cos(2π3ft))+a [low freq signal] (7)

where a is a constant multiplied by the low frequency signal included to simulate the effect of respiratory sinus arrhythmia.

The distal signal (which is delayed) is:

x_{d}(t)=−((sin(2πft−ψ(t)−0.2 sin(2π2ft−ψ(t)−0.2 cos(2π3ft−ψ(t))+b [low freq signal] (8)

where b is a constant multiplied by the low frequency signal included to simulate the effect of respiratory sinus arrhythmia. The distal signal was generated with a known time-varying phase delay of 60 degrees.

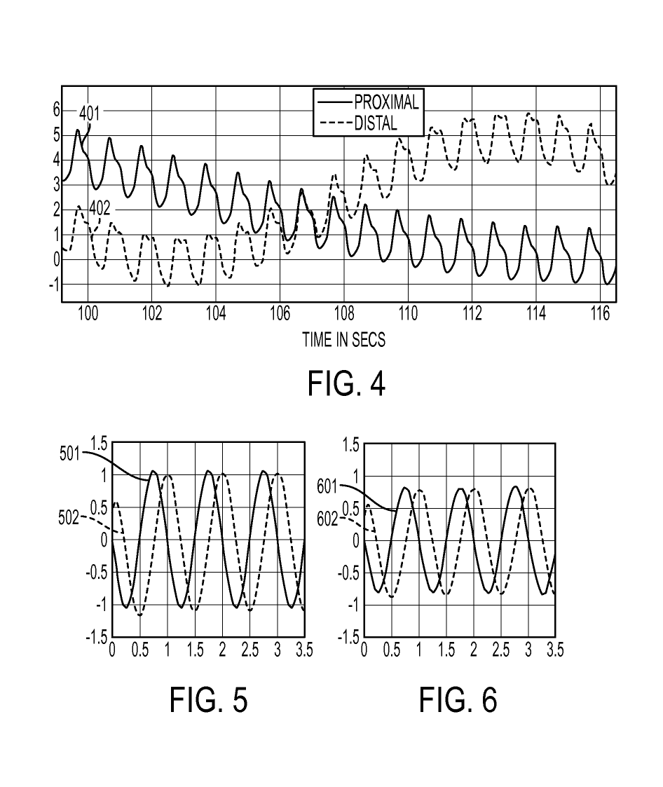

Low frequency modulation was included in each of the proximal and distal signals to simulate the effect of respiration (i.e., respiratory sinus arrhythmia) on cardiovascular signals. FIG. 4 shows simulated proximal and distal waveforms, at 401 and 402, respectively, with a phase delay of approximately 60 degrees. After computing Eqs. (1) through (5), we found the phase difference to be ψ(t)≈60 degrees. This resulted in an estimated PTT of ≈166.7 ms (as expected). FIG. 5 shows the filtered proximal signal and the Hilbert transform of the filtered proximal signal, at 501 and 502, respectively. FIG. 6 shows the filtered distal (delayed) signal and the Hilbert transform of the filtered distal signal, at 601 and 602, respectively. These results demonstrate that PTT can be determined using the methods disclosed herein.

### Various Embodiments

One or more aspects of the present method may be implemented on a dedicated computer system and may also be practiced in distributed computing environments where tasks are performed by remote devices that are linked through a network. The teachings hereof can be implemented in hardware or software using any known or later developed systems, structures, devices, and/or software by those skilled in the applicable art without undue experimentation from the functional description provided herein with a general knowledge of the relevant arts.

One or more aspects of the methods described herein are intended to be incorporated in an article of manufacture, including one or more computer program products, having computer usable or machine readable media. For purposes hereof, a computer usable or machine readable media is, for example, a floppy disk, a hard-drive, memory, CD-ROM, DVD, tape, cassette, or other digital or analog media, or the like, which is capable of having embodied thereon a computer readable program, one or more logical instructions, or other machine executable codes or commands that implement and facilitate the function, capability, and methodologies described herein. Furthermore, the article of manufacture may be included on at least one storage media readable by a machine architecture embodying executable program instructions capable of performing the methodology described in the flow diagrams. The article of manufacture may be included as part of an operating system, a plug-in, or may be shipped, sold, leased, or otherwise provided separately, either alone or as part of an add-on, update, upgrade, or product suite.

It will be appreciated that various of the above-disclosed and other features and functions, or alternatives thereof, may be desirably combined into many other different systems or applications. Various presently unforeseen or unanticipated alternatives, modifications, variations, or improvements therein may become apparent and/or subsequently made by those skilled in the art, which are also intended to be encompassed by the following claims. Accordingly, the embodiments set forth herein are considered to be illustrative and not limiting. Various changes to the above-described embodiments may be made without departing from the spirit and scope of the invention. The teachings of any printed publications including patents and patent applications, are each separately hereby incorporated by reference in their entirety.

What is claimed is:

1. A method for determining arterial pulse wave transit time for a subject, comprising:
receiving a time-series signal for each of a proximal and distal arterial site of a subject's body, said time-series signals being derived from any of: a contact-based photoplethysmographic (PPG) device and from processing image frames acquired by a video imaging device capable of registering a videoplethysmographic (PPG) signal on a least one imaging channel use to acquire that video;
obtaining a proximal and distal analytic signal each comprising a first component that is a waveform of a respective received time-series signal and a second component that is a transform of that waveform;
determining a phase function with respect to time for said first and second components of each of said proximal and distal analytic signals;
subtracting said phase function for said proximal waveform from said phase function for said distal waveform to obtain a phase difference; and
processing said phase difference with said subject's heart rate to determine an arterial pulse wave transit time between said proximal and distal arterial sites.

2. The method of claim 1, wherein said subject's heart rate is extracted from one of said proximal and distal waveforms.

3. The method of claim 1, wherein multiple proximal and distal time-series signals are received.

4. The method of claim 1, wherein, in advance of obtaining any of said proximal and distal analytic signals, further comprising any of:
averaging any of said received proximal or distal time-series signals to obtain a composite proximal or distal time-series signal;
discarding any of said received time-series signals as not being of interest;
weighted averaging on any of said received time-series signals based on a statistical analysis;
detrending any of said received time-series signals to remove non-stationary components;
filtering any of said received time-series signals to restrict frequencies of interest;
performing peak detection on any of said received time-series signals; and
normalizing any of said received time-series signals to have a zero-mean unit variance.

5. The method of claim 1, further comprising analyzing said arterial pulse wave transit time to determine any of:
a blood pressure in said subject's vascular network;
an amount of blood vessel dilation over time;
a blockage of blood flow; and
a blood flow velocity.

6. The method of claim 1, further comprising using said arterial pulse wave transit time to determine an occurrence of any of: cardiac stress, heart disease, and a peripheral vascular disease.

7. The method of claim 1, wherein said transform is a Hilbert Transform.

8. The method of claim 1, wherein said proximal and distal time-series signals are obtained from different devices.

9. The method of claim 1, wherein, in response to said received signals having been captured by different devices, temporally synchronizing said proximal and distal time-series signals.

10. The method of claim 1, wherein said video imaging device is any of: a contact-based video camera, a non-contact-based video camera, a RGB camera, a multi-spectral camera, a hyperspectral camera, and a hybrid camera comprising any combination hereof.

11. The method of claim 1, wherein, in response to said subject's arterial pulse wave transit time not being within a normal range, performing any of: initiating an alert, and signaling a medical professional.

12. The method of claim 1, further comprising communicating said arterial pulse wave transit time to any of: a display device, a storage device, a smartphone, smartwatch, iPad, tablet-PC, laptop, computer workstation, and a remote device over a network.

13. The method of claim 12, wherein said communication comprises any of: text, email, picture, graph, chart, signal, and pre-recorded message.

14. The method of claim 1, wherein said arterial pulse wave transit time is determined in any of: real-time, and on a continuous basis.

15. The method of claim 1, wherein processing said phase difference with said subject's heart rate to determine said arterial pulse wave transit time comprises:
$$PTT = \frac{d φ}{{f}_{HR}}$$
where dφ is said phase difference and f_{HR} is said subject's heart rate in radians per second.

16. A system for determining arterial pulse wave transit time (PTT) for a subject, the system comprising:
a storage device;
a processor in communication with a memory and said storage device, said processor executing machine readable instructions for performing:
receiving a time-series signal for each of a proximal and distal arterial site of a subject's body, said time-series signals being derived from any of: a contact-based photoplethysmographic (PPG) device and from processing image frames acquired by a video imaging device capable of registering a videoplethysmographic (PPG) signal on a least one imaging channel use to acquire that video;
obtaining a proximal and distal analytic signal each comprising a first component that is a waveform of a respective received time-series signal and a second component that is a transform of that waveform;
determining a phase function with respect to time for said first and second components of each of said proximal and distal analytic signals;
subtracting said phase function for said proximal waveform from said phase function for said distal waveform to obtain a phase difference; and
processing said phase difference with said subject's heart rate to determine an arterial pulse wave transit time between said proximal and distal arterial sites.

17. The system of claim 16, wherein said subject's heart rate is extracted from one of said proximal and distal waveforms.

18. The system of claim 16, wherein multiple proximal and distal time-series signals are received.

19. The system of claim 16, wherein, in advance of obtaining any of said proximal and distal analytic signals, further comprising any of:
averaging any of said received proximal or distal time-series signals to obtain a composite proximal or distal time-series signal;
discarding any of said received time-series signals as not being of interest;
weighted averaging on any of said received time-series signals based on a statistical analysis;
detrending any of said received time-series signals to remove non-stationary components;
filtering any of said received time-series signals to restrict frequencies of interest;
performing peak detection on any of said received time-series signals; and
normalizing any of said received time-series signals to have a zero-mean unit variance.

20. The system of claim 16, further comprising analyzing said arterial pulse wave transit time to determine any of:
a blood pressure in said subject's vascular network;
an amount of blood vessel dilation over time;
a blockage of blood flow; and
a blood flow velocity.

21. The system of claim 16, further comprising using said arterial pulse wave transit time to determine an occurrence of any of: cardiac stress, heart disease, and a peripheral vascular disease.

22. The system of claim 16, wherein said transform is a Hilbert Transform.

23. The system of claim 16, wherein said proximal and distal time-series signals are obtained from different devices.

24. The system of claim 16, wherein, in response to said received signals having been captured by different devices, temporally synchronizing said proximal and distal time-series signals.

25. The system of claim 16, wherein said video imaging device is any of: a contact-based video camera, a non-contact-based video camera, a RGB camera, a multi-spectral camera, a hyperspectral camera, and a hybrid camera comprising any combination hereof.

26. The system of claim 16, wherein, in response to said subject's arterial pulse wave transit time not being within a normal range, performing any of: initiating an alert, and signaling a medical professional.

27. The system of claim 16, further comprising communicating said arterial pulse wave transit time to any of: a display device, a storage device, a smartphone, smartwatch, iPad, tablet-PC, laptop, computer workstation, and a remote device over a network.

28. The system of claim 27, wherein said communication comprises any of: text, email, picture, graph, chart, signal, and pre-recorded message.

29. The system of claim 16, wherein said arterial pulse wave transit time is determined in any of: real-time, and on a continuous basis.

30. The system of claim 16, wherein processing said phase difference with said subject's heart rate to determine said arterial pulse wave transit time comprises:
$$PTT = \frac{d φ}{{f}_{HR}}$$
where dφ is said phase difference and f_{HR} is said subject's heart rate in radians per second.
